(19) United States
(12) Patent Application Publication
(10) Pub. No.: US 2012/0330357 A1
(43) Pub. Date: Dec. 27, 2012

(54) MULTI-LOOP ADJUSTABLE KNOTLESS ANCHOR ASSEMBLY AND METHOD FOR REPAIR

(76) Inventor: Raymond Thal, McLean, VA (US)

(21) Appl. No.: 13/531,977

(22) Filed: Jun. 25, 2012

Related U.S. Application Data
(60) Provisional application No. 61/500,657, filed on Jun. 24, 2011.

(51) Int. Cl. A61B 17/04

(52) U.S. Cl. 606/232

(57) ABSTRACT

A suture anchor assembly with an adjustable suture loop for securing a bone mass to soft tissue. The suture anchor system achieves secure, tensioned attachment of soft tissue to a bone mass using a suture anchor assembly in conjunction with an adjustable suture loop composed of loop member with a tensioning suture component extending therefrom and coupled thereto through the creation of a pre-tied slip (or sliding) knot.

### CROSS REFERENCE TO RELATED APPLICATION

This application claims the benefit of U.S. Provisional Patent Application Ser. No. 61/500,657, entitled “MULTI-LOOP ADJUSTBALE KNOTLESS ANCHOR ASSEMBLY AND METHOD FOR REPAIR”, filed Jun. 24, 2011.

### BACKGROUND OF THE INVENTION

1. Field of the Invention

The present invention relates to devices or methods used in tissue repair, more particularly, devices and methods for attachment of biological tissue (i.e., tendons or ligaments) to a bone mass.

2. Description of the Related Art

Soft tissues, such as tendons and ligaments, generally are attached to bone by small collagenous fibers. These connections are strong but permit the tendons and ligaments to be flexible. When a tissue, or a portion of a tissue, is torn away from the bone and requires repair, a surgeon is often required to repair the detached soft tissue with sutures, which are passed through bone tunnels and tied. A number of devices have been developed for securing a ligament or tendon to a bone mass. These devices can be used in place of bone tunneling techniques. These attachment devices are usually anchor element through extensive surgical incisions and, in some circumstances, by arthroscopic surgical techniques. The placement of bone tunnels for repair can be difficult and generally require large open incisions. Recently, through the advent of arthroscopic surgery, where the surgeon looks into a joint cavity with an arthroscope, there has been a trend to repair soft tissues back to bone through small incisions called portals. The unique free loop knotless suture anchor assemblies described herein facilitate this difficult and precise procedure.

A variety of devices are available for attaching objects to bone, such as screws, staples, cement, suture anchors, and sutures alone. These devices have been used to attach soft tissue, such as ligaments, tendons, muscles, as well as objects such as prostheses, to bone. A suture anchor assembly is a device, which utilizes small anchors with suture materials attached thereto. A device, such as a screw, is inserted into the bone mass and anchored in place. After insertion of the anchor, the attached suture is passed through the tissue to be repaired. The tying of a knot in the suture is then required to secure the tissue to the bone. The process of passing the anchored suture through the soft tissue and tying a knot is time consuming and difficult to undertake in the tight space encountered during arthroscopic surgery and sometimes even in conventional open surgery.

Knotless anchor assemblies have been popular and are embodied in a number of prior patents such as U.S. Pat. No. 6,045,574 wherein there is provided an assembly with an anchor means having a snag means, and a hollow sleeve element with a loop suture element attached thereto, wherein the snag means captures a loop suture element of the hollow sleeve element to draw tissue into secure attachment with a bone mass.

However, difficulties still exist and the present invention attempts to address these with a method and apparatus for knotless suture anchoring.

### SUMMARY OF THE INVENTION

It is, therefore, an object of the present invention to provide a suture anchoring system including an anchor having a distal portion and a proximal portion and having side slots extending from the proximal portion of the anchor to an opening located at a location between the proximal portion and the distal portion of the anchor. The suture anchoring system also includes an anchor suture loop for engagement with a suture rod and a tissue suture loop.

It is also an object of the present invention to provide a suture anchoring system wherein the tissue suture loop includes a pre-tied sliding knot with a tensioning suture component extending therefrom.

It is another object of the present invention to provide a suture anchoring system wherein the anchor suture loop includes a pre-tied sliding knot with a tensioning suture component extending therefrom.

It is a further object of the present invention to provide a suture anchoring system wherein the anchor suture loop is secured to a first end of the suture rod.

It is also an object of the present invention to provide a method for securing tissue including the following steps: passing an anchor suture loop through an opening in a suture rod; passing a tissue suture loop through the tissue such that opposed first and second loop sections are formed; passing the anchor suture loop through first and second openings respectively defined by the opposed first and second loop sections; passing the suture rod into an opening of an anchor, the anchor having recesses along its length extending from the opening; capturing the anchor suture loop after passing through the first and second openings in a second opening on the suture rod; and securing the anchor to a desired anchor hole in an anatomical site.

It is another object of the present invention to provide a method including the step of tensioning either the tissue suture loop or the anchor suture.

It is a further object of the present invention to provide a method including the step of tensioning the tissue suture loop.

The present invention allows for the anchoring and soft tissue attachment with limited regard for the depth of the drilled hole or the depth of the bone anchor within the bone mass. This results from the ability to draw the soft tissue toward the bone anchor and bone mass, under the control of the adjustable suture loop, in particular pulling of the tensioning suture component which results in a reduction in the size of the loop member. Because the size of the loop member dictates how far the soft tissue is pulled toward the bone mass, a medical practitioner can readily control the position of the soft tissue relative to the bone mass. The unique use of the suture rod and having the suture loop reside within recesses in the anchor enables a better repair and result without breakage of the suture due to bone abrasion on the suture.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a perspective view of the suture anchor.

FIGS. 2, 3 and 4 are respectively a front plan view, a side plan view and perspective view of a suture rod.

FIGS. 5A-5C and 6A-6D show alternate embodiments for securing an anchor suture loop to the suture rod shown in FIGS. 2, 3 and 4.

FIGS. 7, 8, 9 and 10 show the steps of the present method as applied using the suture anchor assembly in accordance with the present invention.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

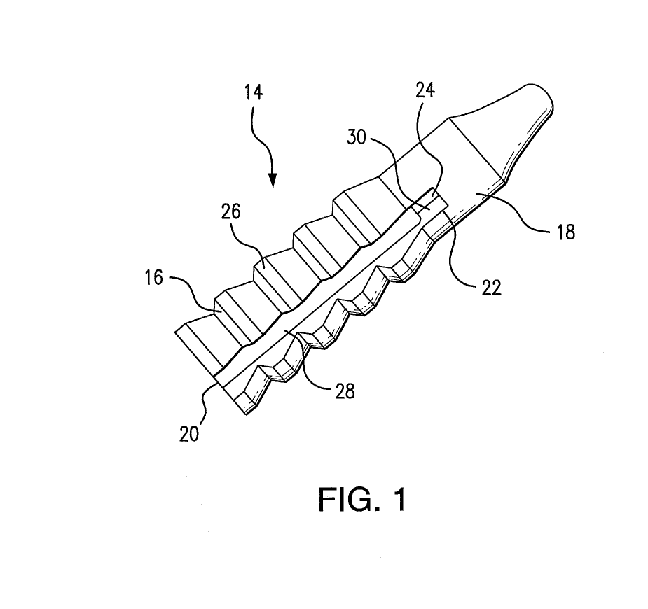

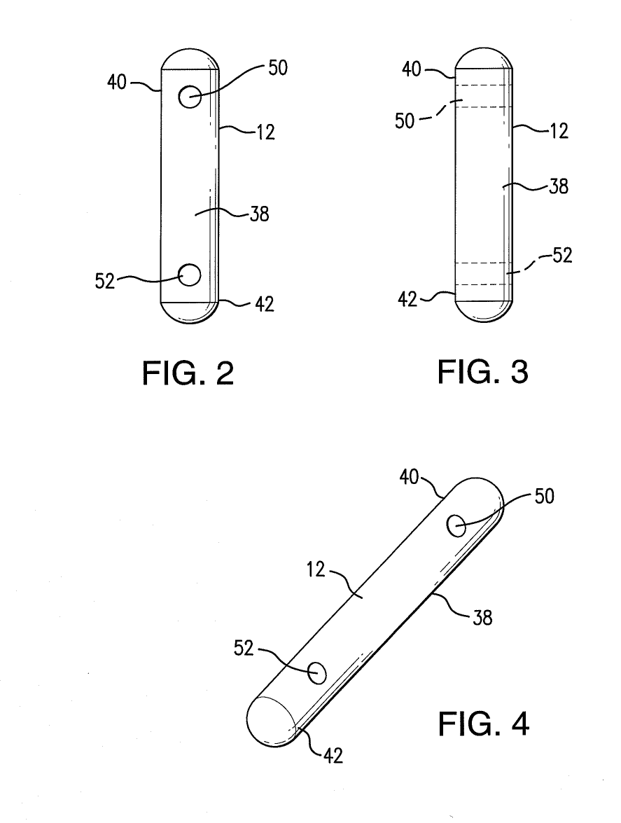

In accordance with the present invention, and with reference to FIGS. 1 to 10, the present procedure is achieved using a bone anchor assembly 10 composed of a suture rod 12 and a suture anchor 14 having a transverse through-hole as will be appreciated based upon the following disclosure. In accordance with a preferred embodiment, the suture anchor 14 is similar to the BIORAPTOR™ suture anchor available from Smith & Nephew. The BIORAPTOR™ suture anchor is disclosed in U.S. Published Patent Application No. 2009/0112270, entitled “ANCHOR ASSEMBLY”, filed Oct. 27, 2008, which is incorporated by reference.

The suture anchor 14 includes a proximal portion 16, a distal portion 18, and an inner cavity 20, if desired. An opening to the inner cavity 20 is located at the proximal portion 16, or at any desired location along the length of the suture anchor 14. A transverse through-hole 22 is located between the proximal portion 16 and the distal portion 18 and extends through the suture anchor 14. Openings 30 are located at each end of the transverse through-hole 22. Located below each opening 30 is a protrusion 24. The protrusions 24 facilitate creation of a path in the wall of a bone hole when the suture anchor 14 is inserted into bone hole. The outer surface of the proximal portion 16 also includes barbs 26, if desired, for substantially reducing the possibility of removal of the suture anchor 14 when inserted into bone, as will be further described below. Any method for securing the anchor to a bone mass can be used. The outer surface also includes slots 28 extending from the openings 30 of the transverse through-hole 22 to the proximal portion 16 of the suture anchor 14. The slots 28 intersect the barbs 26 and are configured for housing of an anchor suture loop 44 after positioning of the suture anchor 14 in bone, as further described below. If the cavity 20 is utilized, it extends into the transverse through-hole 22 and includes a proximal portion and a distal portion for receipt of an insertion member as described in the '270 patent publication. If desired, the distal portion, for receipt of the insertion member, may be threaded. Alternatively, the insertion member can attach outside the suture anchor 14 in the case of a solid anchor.

Referring now to the suture rod 12, the suture rod 12 is composed of a rod body 38 having a first end 40 and a second end 42. A suture element, in the form of an anchor suture loop 44 is provided and can be fixedly secured to the suture rod 12 as discussed below in greater detail.

The suture rod 12 includes an elongated rod body 38. In accordance with a preferred embodiment, the elongated rod body 38 is preferably cylindrical in shape and includes a rounded first end 40 and a rounded second end 42. In certain applications, a rectangular shaped rod can be used. The elongated rod body 38 includes spaced first and second apertures 50, 52. The first and second apertures 50, 52 are preferably positioned adjacent the first end 40 and the second end 42 of the elongated rod body 38, respectively. It is appreciated the position of the first and second apertures 50, 52, as well as the length of the suture rod 12, is such that the first and second apertures 50, 52 extend beyond the openings 30 of the transverse through-hole 22 such that the first and second apertures 50, 52 may be accessed for attachment of the anchor suture loop 44 to the suture rod 12 in the manner discussed below in greater detail.

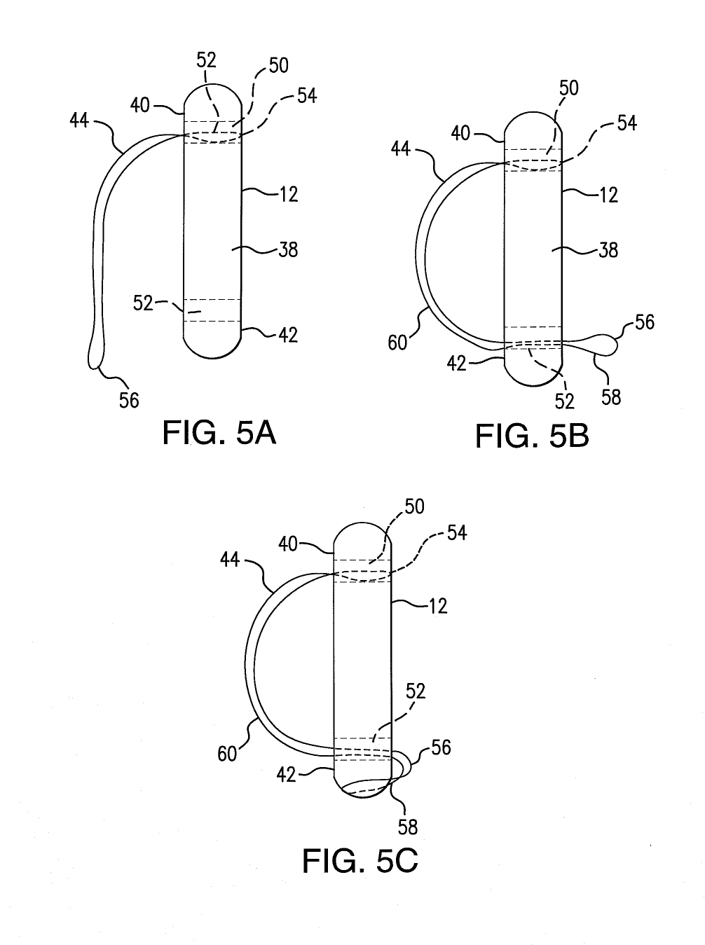

The anchor suture loop 44 is coupled to the suture rod 12 by controlled entanglement with the first and second apertures 50, 52 and the elongated rod body 38 of the suture rod 12. For example, and in conjunction with a first manner of attachment as shown in FIGS. 5A-5C, the anchor suture loop 44 is fixedly secured at the first aperture 50. Considering such an attachment mechanism, the anchor suture loop 44 may be thought of as including a fixed (or first) end 54 directly secured at the first aperture 50 of the suture rod 12 and a free (or second) end 56 that may be freely manipulated for passage through the second aperture 52. When it is desired to entangle the anchor suture loop 44 with the adjustable suture loop 62 as discussed below in greater detail, the free end 56 of the anchor suture loop 44 is passed through the second aperture 52 creating an entanglement loop 58 at the second aperture 52 opposite the entry point for the free end 56 of the anchor suture loop 44, as well as an engagement loop 60 between the first aperture 50 and the second aperture 52. The entanglement loop 58 is then looped around the elongated rod body 38 adjacent the second end 42 of the suture rod 12 effectively locking the anchor suture loop 44 in position when tension is applied to the engagement loop 60 formed by the anchor suture loop 44 between the first aperture 50 and the second aperture 52.

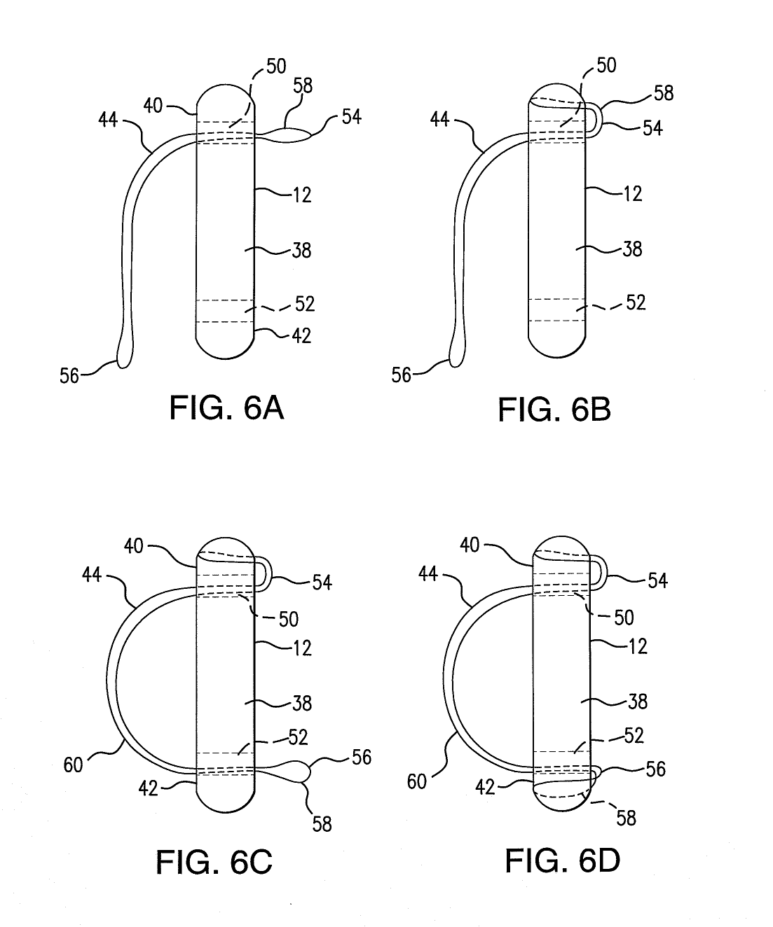

In accordance with an alternate attachment mechanism as shown with reference to FIGS. 6A-6D, the anchor suture loop 44 may be initially separate from the suture rod 12 and secured thereto by looping the anchor suture loop 44 over the elongated rod body 38 at both the first end 40 and the second end 42 of the suture rod 12. Considering the anchor suture loop 44 as being drawn taut and, therefore, including a first suture end 54 and a second suture end 56, the first suture end 54 is passed through the first aperture 50 creating an entanglement loop 58 at the first aperture 50 opposite the entry point for the first suture end 54 of the anchor suture loop 44. The entanglement loop 58 is then looped around the elongated rod body 38 adjacent the first end 40 of the suture rod 12 effectively locking the anchor suture loop 44 in position when tension is ultimately applied to engagement loop 60 formed by the anchor suture loop 44 between the first aperture 50 and the second aperture 52. The second suture end 56 is then passed through the second aperture 52 creating an entanglement loop 58 at the second aperture 52 opposite the entry point for the second suture end 54 of the anchor suture loop 44. The entanglement loop 58 is then looped around the elongated rod body 38 adjacent the second end 42 of the suture rod 12. The application of tension effectively lock the anchor suture loop 44 in position when tension is ultimately applied to engagement loop 60 formed by the anchor suture loop 44 between the first aperture 50 and the second aperture 52, that is, the entanglement loops 58 at the first and second ends 40, 42 of the suture rod 12 wrap thereabout in a manner gripping the suture rod 12.

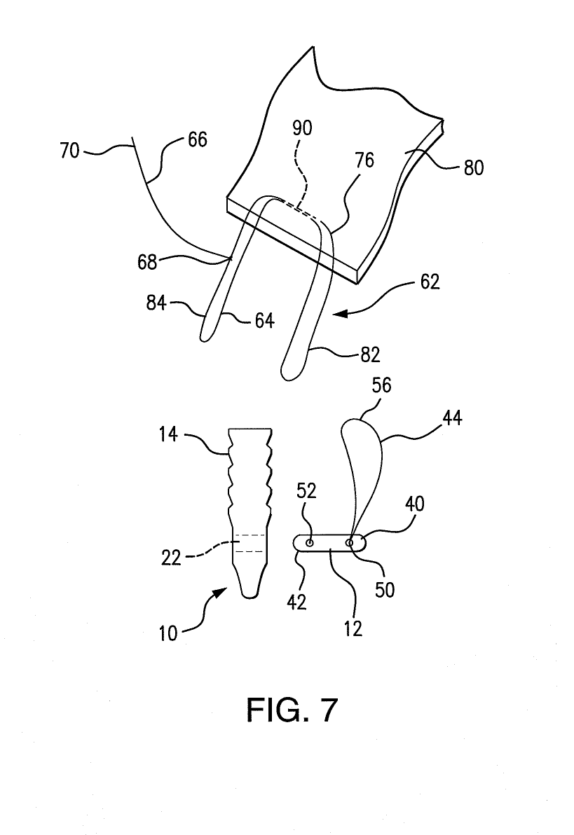

In accordance with a preferred embodiment, the suture anchor assembly 10 is used in conjunction with an adjustable suture loop 62 for securing a bone mass to soft tissue. The present invention achieves secure, tensioned attachment of soft tissue 80 to a bone mass using a suture anchor assembly 10 as described above in conjunction with an adjustable suture loop 62 composed of loop member 64 with a tensioning suture component 66 extending therefrom and coupled thereto through the creation of a slip (or sliding) knot, in particular, a sliding, locking knot 68. The adjustable suture loop 62 is preferably flexible and/or inelastic, and may be prepared with a pretied sliding knot. The free end of the adjustable suture loop 62, that is, the second end 70 of the tensioning suture component 66, is left in place to allow for additional tensioning as is discussed below in greater detail. It is appreciated, the tensioning suture component 66, as shown in FIGS. 7 through 10, can be composed of more than one suture and may have multiple free ends.

The adjustable suture loop 62 is composed of one or more strands of suture. The adjustable suture loop 62 includes a first segment and a second segment tied so as to form a loop member 76 and one or more tensioning suture components 66 extending from the loop member 76. As such, the tensioning suture component 66 may be composed of more than one suture depending upon the number of strands of suture used in the construction of the adjustable suture loop 62. The tensioning suture component 66 includes a first end 78 and a second end 70. The first end 78 is secured to the loop member 76 at the sliding, locking knot 68 defining the loop member 76, while the second end 70 freely extends therefrom for manipulation by the medical practitioner as discussed below in greater detail.

The adjustable suture loop 62 is constructed by tying the first segment and the second segment of the strand of suture with a sliding, locking knot 68. As is appreciated, various knot types are known and a sliding, locking knot is formed in the manner disclosed in Arthroscopic Knot Tying, Frank G. Alberta, et al. (Arthroscopic Knot Tying Chapter 4: pp. 29-38); Arthroscopic knot-tying techniques, Eric R McMillan et al. (An Atlas of Shoulder Arthroscopy Imhoff AB Tucker IB Eu EH (eds) pp 81-95); and Arthroscopic Knots: Determining the Optimal Balance of Loop Security and Knot Security, Ian K. Y. Lo, et al. (The Journal of Arthroscopic and Related Surgery, Vol 20, No 5 (May-June). 2004: pp. 489-502). By constructing the adjustable flexible suture loop 62 with a sliding, locking knot 68, one can alter the size of the loop member 76 by pulling upon the tensioning suture component 66, which functions by pulling a portion of the suture strand 70 through the sliding, locking knot 68 and ultimately reducing the size of the loop member 64 while increasing the length of the tensioning suture component 66.

In accordance with the present method, the adjustable suture loop 62 is first passed through the soft tissue 80 one wishes to secure to a bone mass. Referring to FIG. 7, the adjustable suture loop 62, in particular, the loop member 64 thereof, is drawn through the soft tissue 80 with a surgical needle. A variety of suture passing methods can be used such as arthroscopic suture passers, arthroscopic suture shuttling devices or suture, or the like. The loop member 64 is passed through the soft tissue 80, one or more times, such that opposed first and second loop sections 82, 84 are formed when the loop member 64 is effectively split into two sections by the soft tissue 80; that is, with the soft tissue 80 between the opposed loop sections 82, 84. In particular, the loop member 64 is pulled through the soft tissue 80 until the two loop sections 82, 84 are of substantially the same size and in alignment. It is appreciated that the loop member 64 is relatively long so that the opposed loop sections 82, 84 may be brought outside of the joint under repair. As shown in FIG. 7, the tensioning suture component 66 of the adjustable suture loop 62 also extends from one of the loop sections 82 and is similarly accessible from outside of the joint under repair.

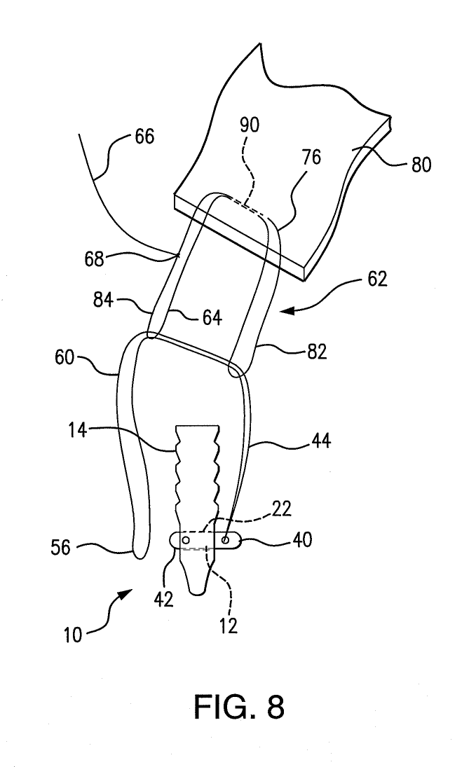

With the loop sections 82, 84 of the adjustable suture loop 62 outside of the joint, and with reference to FIG. 8, and the suture rod 12 positioned within the transverse through-hole 22 with the first and second apertures 50, 52 extending beyond the exterior surface of the suture anchor 14, aligning with the slots 28 of the anchor 14, the anchor suture loop 44 is passed through the openings 86, 88, respectively, defined by the opposed first and second loop sections 82. That is, and considering the attachment mechanism disclosed above with reference to FIG. 8, the free end 56 of the anchor suture loop 44 is drawn through the openings 86, 88 such that the anchor suture loop 44 is intertwined with the opposed loop sections 82, 84. In this arrangement, the central portion 90 of the loop member 64, between the loop sections 82, 84, is in direct contact with the soft tissue 80 securing the anchor suture loop 44 to the soft tissue 80 such that the loop sections 82, 84 may simultaneously pull against the anchor suture loop 44 without fear that the loop member 64 will become disengaged with the soft tissue 80.

Thereafter, the free end 56 of the anchor suture loop 44 is captured at the second end 42 of the suture rod 12 through the formation of an entanglement loop 58 which is secured at the second end 42 of the suture rod 12 in the manner discussed above with reference to FIGS. 5A-5C and 6A-6D; that is, the free end 56 of the anchor suture loop 44 is captured, entangled, coupled to, or otherwise attached to the second end 42 of the suture rod 12 for manipulation of the anchor suture loop 44.

It is appreciated the attachment mechanism disclosed with reference to FIGS. 6A-6D may also be employed. The only difference between this attachment mechanism and that disclosed with reference to FIGS. 5A-5C involves the need to attach the first end 54 of the anchor suture loop 44 to the first end 40 of the suture rod 12 prior to attachment at the second end 42 of the suture rod 12.

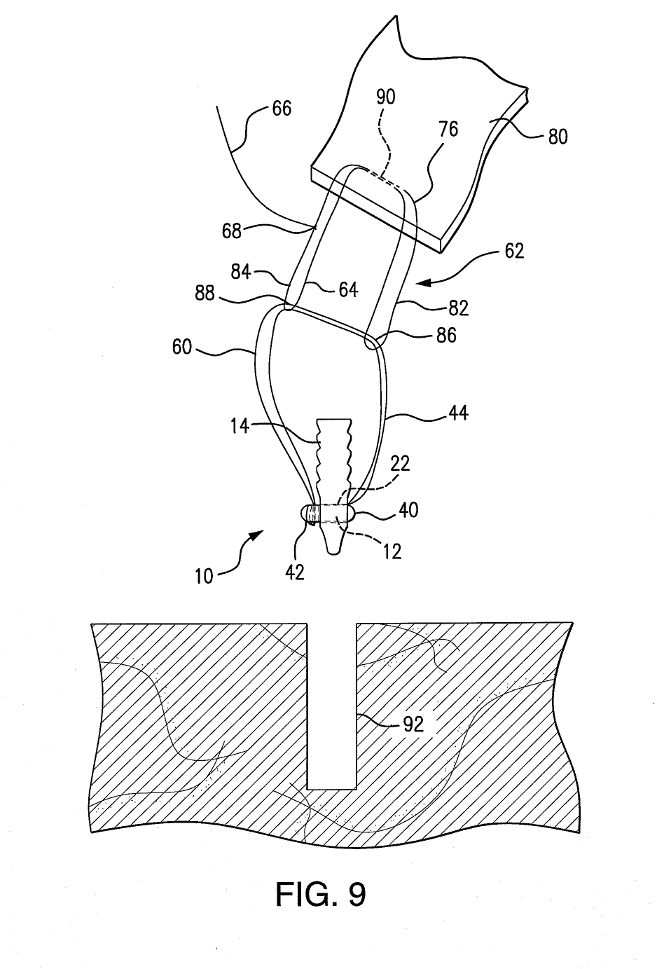

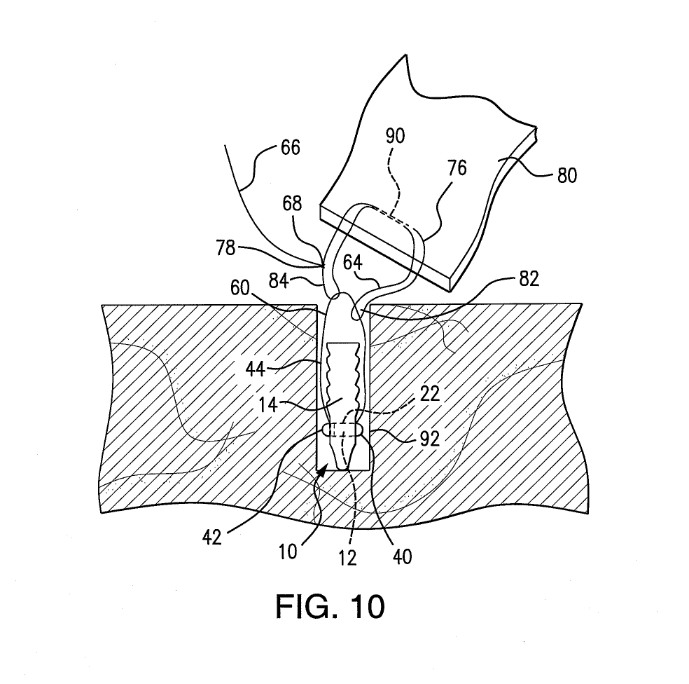

With the anchor suture loop 44 passed through the openings 86, 88 defined by the opposed first and second loop sections 82, 84 and the ends 54, 56 of the anchor suture loop 44 secured to the first end 40 and second end 42 of the suture rod 12, the anchor suture loop 44 is linked to the loop member 64 and ultimately the soft tissue 80 (see FIG. 9). Referring to FIG. 10, the bone anchor assembly 10 is then inserted within an anchor hole 92 preferably predrilled in the bone mass. In accordance with the disclosed embodiment, the bone anchor assembly 10 is inserted into the hole 92 securing the two ends 54, 56 of the anchor suture loop 44 in the bone mass. When inserting the bone anchor assembly 10 within the hole 92, care should be taken to secure the anchor suture loop 44 extending along the sides of the suture anchor 14 in position in the slots 28 of the bone anchor 14. This will help prevent unnecessary wear on the anchor suture loop 44 by preventing interaction with the bone mass that might abrade the suture material. It is appreciated the bone hole may be lined with a sleeve into which the bone anchor assembly is secured upon deployment.

With the bone anchor assembly 10 securely held within the drill hole 92, the loop member 64 is tensioned so as to reduce the size of the loop member 64 and draw the soft tissue 80 toward the bone mass. In particular, the tensioning suture component 66 is pulled in a direction away from the loop member 64 causing the suture strand to be drawn through the sliding, locking knot 68 reducing the size of the loop member 64 and consequently drawing the soft tissue 80 toward the bone mass since the size of the anchor suture loop 44 is fixed. The tensioning suture component 66 is pulled and the loop member 64 size is reduced until such a time that the soft tissue 80 is fully pulled toward the bone mass. The procedure may be repeated depending upon the needs of the procedure. The excess suture material of the tensioning suture component(s) 66 may then be cut away and the incision closed. With some anchor member designs, additional tensioning can be achieved by pushing the anchor deeper into the drill hole after the loop is tensioned by the sliding knot.

It is also appreciated the present invention allows for anchoring and soft tissue attachment with limited regard for the depth of the drilled hole or the depth of the bone anchor within the bone mass. This results from ability to draw the soft tissue toward the bone anchor and bone mass under the control of the adjustable suture loop, in particular pulling of the tensioning suture component which results in a reduction in the size of the loop member. Because the size of the loop member dictates how far the soft tissue is pulled toward the bone mass, a medical practitioner can readily control the position of the soft tissue relative to the bone mass.

In many situations throughout the discussion above, the terminology relating to the secure attachment of soft tissue to bone mass has been used. Such terminology refers to the attachment or reattachment of tissue to a bone mass by securely binding the tissue to the bone mass utilizing the novel knotless suture anchor assembly 10. The suture element can be made up of a known suture material, or it can be made of polymer materials, or can be formed of bioabsorbable material such as a polylactide polymer.

While the preferred embodiments have been shown and described, it will be understood that there is no intent to limit the invention by such disclosure, but rather, is intended to cover all modifications and alternate constructions falling within the spirit and scope of the invention.

I claim:

1. A suture anchoring system, comprising:
an anchor having a distal portion and a proximal portion and having side slots extending from the proximal portion of the anchor to an opening located at a location between the proximal portion and the distal portion of the anchor;
an anchor suture loop for engagement with a suture rod; and
a tissue suture loop.

2. The suture anchoring system according to claim 1, wherein the tissue suture loop includes a pre-tied sliding knot with a tensioning suture component extending therefrom.

3. The suture anchoring system according to claim 1, wherein the anchor suture loop includes a pre-tied sliding knot with a tensioning suture component extending therefrom.

4. The suture anchoring system according to claim 1, wherein the anchor suture loop is secured to a first end of the suture rod.

5. A method for securing tissue, comprising:
passing an anchor suture loop through an opening in a suture rod;
passing a tissue suture loop through the tissue such that opposed first aid second loop sections are formed;
passing the anchor suture loop through first and second openings respectively defined by the opposed first and second loop sections;
passing the suture rod into an opening of an anchor, the anchor having recesses along its length extending from the opening;
capturing the anchor suture loop after passing through the first and second openings in a second opening on the suture rod; and
securing the anchor to a desired anchor hole in an anatomical site.

6. The method according to claim 5, further including the step of tensioning either the tissue suture loop or the anchor suture.

7. The method according to claim 5, further including the step of tensioning the tissue suture loop.
